(19) United States
(12) Patent Application Publication
(10) Pub. No.: US 2022/0015665 A1
(43) Pub. Date: Jan. 20, 2022

(54) Multipolar Cannula

(71) Applicant: Pajunk GmbH Medizintechnologie, Geisingen (DE)

(72) Inventors: Simone Pajunk-Schelling, Geisingen (DE); Martin Hauger, Donaueschingen (DE)

(73) Assignee: Pajunk GmbH Medizintechnologie, Geisingen (DE)

(21) Appl. No.: 17/295,997

(22) PCT Filed: Nov. 12, 2019
(86) PCT No.: PCT/EP2019/081060

§ 371 Date: May 21, 2021

(30) Foreign Application Priority Data
Nov. 23, 2018 (DE) 10 2018 129 541.3

(51) Int. Cl. A61B 5/0538; A61B 5/00

(52) U.S. Cl. CPC A61B 5/0538; A61B 2562/125; A61B 5/7235; A61B 5/6848

(57) ABSTRACT

A multi-polar cannula having a cannula tube with a distal end and a proximal end and with a first electrode and at least one second electrode wherein the cannula tube has a cannula tube body and a layer that electrically insulates the first and second electrodes from each other, wherein the distal end of the cannula tube has a distal tip, wherein the first electrode is formed by the cannula tube body, and wherein the first electrode and the second electrode are connectable to a bioimpedance meter.

The invention relates to a multipolar cannula.

Known are multipolar cannulas, for example bipolar cannulas with a first electrode and a second electrode that are implemented such that they are electrically insulated with respect to one another. A known structure comprises sliding onto [an electrically conducting] cannula tube body an electrically insulating layer in the form of a tube of insulating synthetic material and to slide a further electrically conducting tube body onto the electrically insulating tube.

The invention therefore addresses the problem of further developing a multipolar cannula.

The problem addressed by the invention is resolved by a multipolar cannula with the characteristics of Patent claim 1.

Advantageous embodiments and further developments of the invention are specified in the dependent claims.

The multipolar cannula according to the invention comprises a cannula tube having a distal end and a proximal end and a first electrode and at least one second electrode, wherein the cannula tube comprises a cannula tube body and a coating layer electrically insulating the first and the second electrode with respect to one another, wherein the distal end of the cannula tube comprises a distal tip, wherein the first electrode is formed by the cannula tube body and wherein the first electrode and the second electrode are connectable to a bio-impedance measuring unit.

Through such an implementation a compactly structured multipolar cannula is provided with which bio-impedance measurements can be carried out. For a bio-impedance measurement the electrical impedance between the free ends of the first and second electrode is determined.

At the proximal end of the cannula tube an extension is disposed comprising an electrically contacting connection for the electrodes. Thereby in simple manner an electrical contacting of the electrodes can be achieved, in particular if the electrodes extend over the entire length of the cannula tube from the distal end up to the electrically contacting connection.

According to an especially preferred further development of the invention, the first electrode and the second electrode are optionally connectable across a switch to a power supply or to the bio-impedance measuring unit. When the two electrodes are connected to the bio-impedance measuring unit, it is feasible to determine in what type of tissue the tip of the multipolar cannula is located at any given time. The two electrodes can, on the other hand, be utilized within the framework of the multipolar cannula for stimulation by means of appropriate stimulation loading.

The multipolar cannula preferably comprises an analysis unit or is connected to an analysis unit which is developed to analyze the electric signals from the electrodes and to generate a display signal, for example in the form of an acoustic or optical display signal. The display signals can be developed such that a user can detect the type of tissue in which the tip of the multipolar is located at any given time. The electrically insulating coating layer and at least the second electrode are applied onto the cannula tube body using a thin film process. The electrically insulating coating layer is thereby developed in particular as an electrically insulating film. Thereby significantly lesser cross sections of the cannula are enabled in comparison to conventional cannulas in the form of a double tube.

According to an especially preferred embodiment, the electrically insulating layer has a thickness of a few micrometers, preferably a thickness of less than 1 micrometer. The outer dimensions in the cross section of the multipolar cannula can thereby be significantly reduced.

The second electrode has preferably a thickness of a few micrometers, preferably a thickness of less than 1 micrometer. The diameter of the multipolar cannula can thereby be markedly reduced.

According to a preferred embodiment, the electrically insulating layer is comprised of parylene. Parylenes are suitable for the surface coating onto the most diverse substrate materials and for surface coating of the most diverse geometric objects such that they are especially suitable for coating cannula tube bodies.

Except for the distal tip, the electrically insulating layer preferably covers a distal segment of the cannula tube body or substantially completely. Thereby good insulation can be enabled between the cannula tube body and the second electrodes applied in or on the insulating layer.

The second electrode is advantageously applied onto the electrically insulating layer using a thin film process whereby a minimal layer thickness of the second electrode can be realized.

It is especially preferred for the second electrode to be comprised of aluminum since aluminum has good electrical conductivity and, furthermore, adheres well on different materials such as, for example, parylenes.

The second electrode is advantageously spaced apart from the distal end of the electrically insulating layer and, in particular, covers the electrically insulating layer except for a distal annularly circumferential segment. Due to the spacing from the distal end of the electrically insulating layer, good electrical insulation can be enabled between the second electrode and the cannula tube body. If the second electrode covers the electrically insulating layer except for a distal annularly circumferential segment, a large-area second electrode with good electrically conducting properties can be provided.

On the second electrode at least in segments a second electrically insulating layer is advantageously disposed.

The second electrically insulating layer is advantageously comprised of parylenes or white lacquer. Especially in the case in which the second electrode is fabricated of aluminum, it is useful to utilize as the second electrically insulating layer a white lacquer in order to least impair the conductivity of the aluminum layer.

The second electrically insulating layer preferably covers the second electrode except for at least a distally disposed active segment to enable the safe manipulation of the cannula by a user. It is feasible for each of the second electrodes to comprise more than one active segment whereby complex geometries of electrode structures are enabled.

An especially preferred embodiment of the invention provides for the second electrode to be disposed in the electrically insulating layer. Such disposition can be attained thereby that the second electrode and the electrically insulating layer are applied jointly onto the cannula tube body. This enables embedding the second electrode or also several second electrodes into the electrically insulating layer.

The invention will be explained in detail in conjunction with the following Figures. Therein depict:

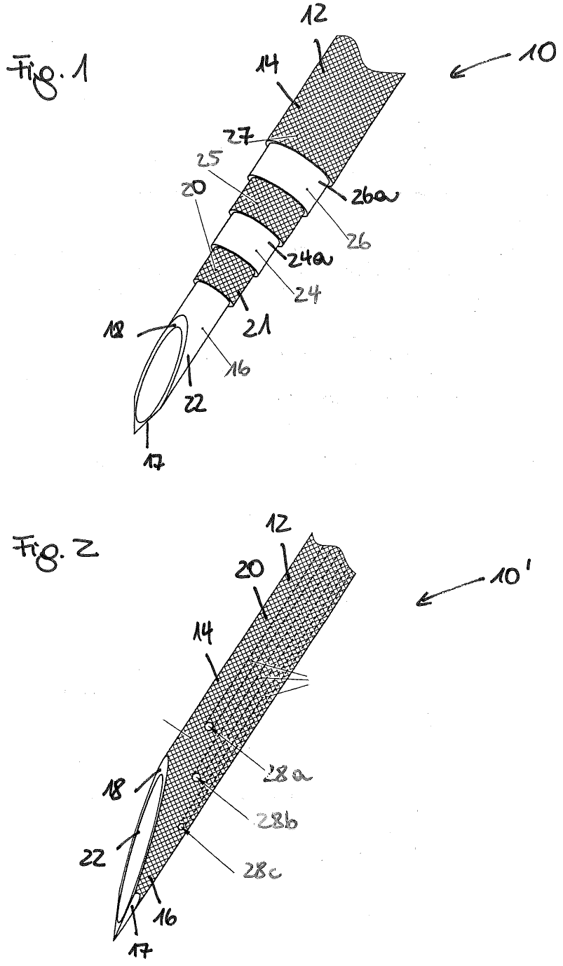

FIG. 1: a schematic perspective representation of a distal end of a first embodiment example of a multipolar cannula according to the invention and

FIG. 2: a schematic perspective representation of a distal end of a second embodiment example of a multipolar cannula according to the invention.

FIG. 1 shows a first embodiment example of a multipolar cannula 10 with a cannula tube 12 having a distal end 14 and a not shown proximal end. The cannula tube 12 comprises a cannula tube body 18 and an electrically insulating layer 20. The cannula tube body 18 forms a first electrode 22. The cannula tube body 18 is therefore fabricated of an electrically conducting material and developed, for example, as a steel tube.

At its distal end 14 the cannula tube body 18 comprises a distal tip 16 which can, for example, be formed thereby that the distal end 14 extends obliquely at an angle, for example at an angle of approximately 45°, with respect to the longitudinal axis of the cannula tube 12. The distal end of the distal tip 16 can additionally comprise a facet cut 17 in order to enhance the sharpness of the distal tip 16.

The electrically insulating layer 20 can be applied in a thin film process as an electrically insulating surface coating and covers, in particular circumferentially, wherein the distal tip 16 can remain exposed. The electrically insulating layer 20 can be developed up to the proximal end of the cannula tube body 18.

Disposed onto the electrically insulating layer 20 is a second electrode 24 which is, for example, disposed such that it encircles the electrically insulating layer 20 such that the distal end of the second electrode 24 is spaced apart from the distal end 14 of the electrically insulating layer 20 and, in particular, an annularly circumferential segment 21 of the electrically insulating layer 20 remains exposed. Through the circumferential segment 21 sufficient electrical insulation between the first electrode 22 and the second electrode 24 is ensured even at the active surfaces that remain exposed. The second electrode 24 can herein extend up to the proximal end of the cannula tube 12.

On the second electrode 24 a second electrically insulating layer 25 is disposed, in particular such that the second electrically insulating layer 25 covers the second electrode 24 except for at least one distally disposed active segment 24a. The active segment 24a can be developed for example to be annularly circumferential or it can assume nearly any desired geometric shape. It can, in particular, be developed as a circular, elliptical or rectangular area.

If further poles are to be provided for an above described multipolar cannula 10, then, as is evident in the embodiment example depicted in FIG. 1, a third electrode 26 can be applied onto the second electrically insulating layer 25, preferably also using a thin film process, which electrode is again also covered with a third electrically insulating layer 27 except for at least one distally disposed active segment 26a.

In the same manner, the cannula can be supplemented with further poles.

At least the first electrode 22 and the second electrode 24 are connected to a bio-impedance measuring unit.

At the proximal end of the multipolar cannula 10 the electrodes 22, 24, 26 can be electrically contacting so as to be conducting such that via the electrodes 22, 24, 26 an electrical stimulation is feasible when the multipolar cannula 10 is introduced into the body of a patient. For this purpose, at the proximal end of the cannula tube an extension can be disposed which comprises an electrically contacting connection for the electrodes 22, 24, 26.

In an embodiment the first electrode 22 and the second electrode 24 are connectable, by means of a not shown switch, to a power supply or to the bio-impedance measuring unit.

The multipolar cannula can comprise an analysis unit or be connected to an analysis unit which is developed to analyze the electric signals from the electrodes and to generate a display signal, for example in the form of an acoustic or optical display signal.

FIG. 2 shows a further embodiment example of a multipolar cannula 10′ which, like the multipolar cannula 10 according to the first embodiment example, comprises the cannula tube 12 having a distal end 14 and a not shown proximal end and a cannula tube body 18 and an electrically insulating layer 20. The cannula tube body 18 again represents the first electrode 22.

The multipolar cannula 10′ according to the second embodiment example differs from the first embodiment example in that in the electrically insulating layer 20 at least one, in the present embodiment example three, second electrodes 28a, 28b, 28c are embedded. The electrodes 28a, 28b, 28c are developed as track conductors in the electrically insulating layer 20 and extend from the distal region of the cannula tube 12 up to the proximal end. They can reach up to the distal tip 16 of the cannula tube 12. The active regions of electrodes 28a, 28b, 28c can be exposed by removing the electrically insulating layer 20 over the distal ends of electrodes 28a, 28b, 28c. In the embodiment example the electrodes 28a, 28b, 28c are developed as substantially round track conductors running in parallel. However, it is evident that the electrodes can assume manifold geometric forms.

A further difference of the second embodiment example of the multipolar cannula 10′ relative to the first embodiment example is comprised in that the electrical insulating layer 20 covers the entire cannula tube body 18 up to and over the distal tip 16 and only leaves exposed the front face of the cannula tube body 18 as well as optionally present facet cut faces 17.

### LIST OF REFERENCE SYMBOLS

10 Multipolar cannula
10′ Multipolar cannula
12 Cannula tube
14 Distal end
16 Distal tip
18 Cannula tube body
20 Electrically insulating layer
21 Segment
22 First electrode
24 Second electrode
24a Active segment
25 Second electrically insulating layer
26 Third electrode
26a Active segment
27 Third electrically insulating layer
28a Electrode
28b Electrode
28c Electrode

1. A multipolar cannula comprises:
a cannula tube (12), having a distal end and a proximal end, and with a first electrode and at least one second electrode,
wherein the cannula tube comprises a cannula tube body and an insulating layer electrically insulating the first and the second electrode with respect to one another,
wherein the distal end of the cannula tube comprises a distal tip,
wherein the first electrode is formed by the cannula tube body, and
wherein the first electrode and the second electrode are connectable to a bio-impedance measuring unit.

2. The multipolar cannula as in claim 1, wherein an extension is disposed at the proximal end of the cannula tube and the extension comprises an electrically contacting connection for the electrodes.

3. The multipolar cannula as in claim 2, wherein the first electrode and the second electrode extend from the distal end up to the extension.

4. The multipolar cannula as in claim 2, wherein the first electrode and the second electrode are connectable to a power supply or to the bio-impedance measuring unit by a switch.

5. The multipolar cannula as in claim 1, wherein the multipolar cannula connects to an analysis unit which to analyzes signals from the electrodes and generates a display signal.

6. The multipolar cannula as in claim 1, wherein the electrically insulating layer and at least the second electrode are applied onto the cannula tube body using a thin film process.

7. The multipolar cannula as in claim 1 wherein the electrically insulating layer has a thickness of less than one micrometer.

8. The multipolar cannula as in claim 1, wherein the second electrode has a thickness of less than one micrometer.

9. The multipolar cannula as in claim 1, wherein the electrically insulating layer is comprised of parylene.
